(19) United States
(12) Patent Application Publication
(10) Pub. No.: US 2026/0263452 A1
(43) Pub. Date: Sep. 10, 2026

(54) ONCOLOGY COMBINATION THERAPY AND METHODS OF USE

(71) Applicant: BeyondSpring Pharmaceuticals, Inc., Florham Park, NJ (US)

(72) Inventors: Lan Huang, New York, NY (US); Yingjuan June Lu, West Lafayette, IN (US); George Kenneth Lloyd, Redwood City, CA (US)

(21) Appl. No.: 19/489,243

(22) PCT Filed: May 20, 2024
(86) PCT No.: PCT/US2024/030205

§ 371 Date: Dec. 1, 2025

Related U.S. Application Data
(60) Provisional application No. 63/505,980, filed on Jun. 2, 2023.

(51) Int. Cl. A61K 31/496; A61K 31/506; A61P 35/00

(52) U.S. Cl. CPC A61K 31/496; A61K 31/506; A61P 35/00

(57) ABSTRACT

Disclosed herein is the treatment of cancer by co-administering plinabulin and a cyclin-dependent kinase inhibitor, such as abemaciclib.

### INCORPORATION BY REFERENCE TO ANY PRIORITY APPLICATIONS

This application claims priority to U.S. Provisional Application No. 63/505,980, filed Jun. 2, 2023, the content of which is incorporated by reference in its entirety.

### FIELD

The present disclosure relates to the field of chemistry and medicine. More specifically, it relates to combination therapy for the treatment of cancer.

### BACKGROUND

The cyclin-dependent kinase (CDK) family fulfills an essential role in regulating cell division and modulating transcription. These inhibitors interrupt kinase activity, disrupting phosphorylation of the retinoblastoma protein pathway, which arrests the cell cycle in the G1 phase and inhibits cancer progression. Several CDK inhibitors that specifically block the activity of CDK4 and CDK6 have been approved for the treatment of metastatic hormone receptor positive breast cancer.

Despite the success of CDK inhibitor therapies, only 60-70% of all breast cancers respond to CDK inhibitor therapies and many patients without intrinsic resistance eventually acquire resistance. Thus, there exists a need for improved anti-cancer therapies utilizing this treatment modality.

### SUMMARY

Some embodiments described herein include a method of treating cancer, comprising co-administering to a subject in need thereof plinabulin and a cyclin-dependent kinase (CDK) inhibitor. In some embodiments, plinabulin is administered intravenously. In some embodiments, the CDK inhibitor is administered orally. In some embodiments, the CDK inhibitor inhibits cyclin-dependent kinase 4 or cyclin-dependent kinase 6. In some embodiments, the CDK inhibitor is selected from the group consisting of palbociclib, ribociclib, and abemaciclib. In some embodiments, the CDK inhibitor is abemaciclib. In some embodiments, abemaciclib is administered at a dose of 25 mg to 400 mg. In some embodiments, abemaciclib is administered once per day. In some embodiments, abemaciclib is administered twice per day. In some embodiments, the dose of plinabulin administered is from 10 mg/m² to 40 mg/m². In some embodiments, the dose of plinabulin administered is from 15 mg to 96 mg. In some embodiments, the dose of plinabulin administered is about 40 mg. In some embodiments, plinabulin is administered once per week. In some embodiments, plinabulin is administered twice per week. In some embodiments, plinabulin is administered once every three weeks.

Some embodiments described herein include a pharmaceutical composition, comprising plinabulin or a pharmaceutically acceptable salt thereof and a CDK inhibitor or a pharmaceutically acceptable salt thereof. In some embodiments, the CDK inhibitor inhibits cyclin-dependent kinase 4 and/or cyclin-dependent kinase 6. In some embodiments, the CDK inhibitor is selected from the group consisting of palbociclib, ribociclib, and abemaciclib. In some embodiments, the CDK inhibitor is abemaciclib.

Some embodiments described herein include a kit, comprising:
a first pharmaceutical composition comprising plinabulin or a pharmaceutically acceptable salt thereof; and
a second pharmaceutical composition comprising a CDK inhibitor. In some embodiments, the first pharmaceutical composition is in an intravenous formulation and the second pharmaceutical composition is an oral formulation. In some embodiments, the CDK inhibitor inhibits cyclin-dependent kinase 4 and/or cyclin-dependent kinase 6. In some embodiments, the CDK inhibitor is selected from the group consisting of palbociclib, ribociclib, and abemaciclib. In some embodiments, the CDK inhibitor is abemaciclib.

### BRIEF DESCRIPTION OF THE DRAWINGS

FIG. 1 is a graph showing the mean tumor volume in an MDA-MB-231 human breast cancer xenograft model upon administration of vehicle control, abemaciclib alone, plinabulin alone, and the combination of plinabulin and abemaciclib.

FIG. 2 is a graph showing the percentage of tumor growth inhibition in an MDA-MB-231 human breast cancer xenograft model upon administration of vehicle control, abemaciclib alone, plinabulin alone, and the combination of plinabulin and abemaciclib.

### DETAILED DESCRIPTION

Disclosed herein are methods for treating cancer. In some embodiments, the methods include co-administration of plinabulin and a kinase inhibitor. Administration of plinabulin herein includes administering a pharmaceutically acceptable salt or solvate (including hydrate) of plinabulin. In some embodiments, plinabulin is provided as a monohydrate. Administration of the kinase inhibitor herein includes administering a pharmaceutically acceptable salt of the kinase inhibitor.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity.

As used herein, the terms “co-administer,” “co-administering,” or “co-administration,” refers to two or more agents or therapies that have a biological effect on a subject at the same time, regardless of when or how they are actually administered. In one embodiment, the agents or therapies are administered simultaneously. In one such embodiment, administration in combination is accomplished by combining the agents in a single dosage form. In another embodiment, the agents or therapies are administered sequentially. In some embodiments, the administration may be separated by a period of time, for example, 30 minutes, 1 hour, 2 hours, 1 day, 2 days, 3 days, or 1 week. In one embodiment the agents are administered through the same route, such as orally. In another embodiment, the agents are administered through different routes, such as one being administered orally and another being administered i.v.

### Plinabulin

Plinabulin, (3Z,6Z)-3-benzylidene-6-{[5-(2-methyl-2-propanyl)-1H-imidazol-4-yl]methylene}-2,5-piperazinedione, is a synthetic analog of the natural compound phenylahistin. Plinabulin can be readily prepared according to methods and procedures detailed in U.S. Pat. Nos. 7,064,201 and 7,919,497, which are incorporated herein by reference in their entireties.

In some embodiments, plinabulin is administered at a dose in the range of about 1-50 mg/m² of the body surface area. In some embodiments, plinabulin is administered at a dose in the range of about 5 to about 50 mg/m² of the body surface area. In some embodiments, plinabulin is administered at a dose in the range of about 10 to about 40 mg/m² of the body surface area. In some embodiments, plinabulin is administered at a dose in the range of about 15 to about 30 mg/m² of the body surface area. In some embodiments, plinabulin is administered at a dose in the range of about 0.5-1, 0.5-2, 0.5-3, 0.5-4, 0.5-5, 0.5-6, 0.5-7, 0.5-8, 0.5-9, 0.5-10, 0.5-11, 0.5-12, 0.5-13, 0.5-13.75, 0.5-14, 0.5-15, 0.5-16, 0.5-17, 0.5-18, 0.5-19, 0.5-20, 0.5-22.5, 0.5-25, 0.5-27.5, 0.5-30, 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 10-40, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m², of the body surface area. In some embodiments, plinabulin is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, plinabulin is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, or 40 mg/m² of the body surface area. In some embodiments, plinabulin is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, or 50 mg/m² of the body surface area. In some embodiments, plinabulin is administered at a dose of about 10, 13.5, 20, or 30 mg/m² of the body surface area. In some embodiments, plinabulin is administered at a dose of about 20 mg/m² of the body surface area.

In some embodiments, the plinabulin dose is about 5 mg-100 mg, or about 10 mg-80 mg. In some embodiments, the plinabulin dose is about 15 mg-100 mg, or about 20 mg-80 mg. In some embodiments, plinabulin is administered at a dose in the range of about 15 mg-60 mg. In some embodiments, the plinabulin dose is about 0.5 mg-3 mg, 0.5 mg-2 mg, 0.75 mg-2 mg, 1 mg-10 mg, 1.5 mg-10 mg, 2 mg-10 mg, 3 mg-10 mg, 4 mg-10 mg, 1 mg-8 mg, 1.5 mg-8 mg, 2 mg-8 mg, 3 mg-8 mg, 4 mg-8 mg, 1 mg-6 mg, 1.5 mg-6 mg, 2 mg-6 mg, 3 mg-6 mg, or about 4 mg-6 mg. In some embodiments, plinabulin is administered at about 2 mg-6 mg or 2 mg-4.5 mg. In some embodiments, plinabulin is administered at about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-6 0 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-4 8 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 15 mg-120 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, plinabulin dose is greater than about 0.5 mg, 1 mg, 1.5 mg, 2 mg, 3 mg, 4 mg, 5 mg, 6 mg, 7 mg, 8 mg, 9 mg, about 10 mg, about 12.5 mg, about 13.5 mg, about 15 mg, about 17.5 mg, about 20 mg, about 22.5 mg, about 25 mg, about 27 mg, about 30 mg, or about 40 mg. In some embodiments, the plinabulin dose is about less than about 1 mg, 1.5 mg, 2 mg, 3 mg, 4 mg, 5 mg, 6 mg, 7 mg, 8 mg, 9 mg, about 10 mg, about 12.5 mg, about 13.5 mg, about 15 mg, about 17.5 mg, about 20 mg, about 22.5 mg, about 25 mg, about 27 mg, about 30 mg, about 40 mg, or about 50 mg.

In some embodiments, the dose of plinabulin is 40 mg.

In various embodiments, plinabulin is administered once in a kinase inhibitor treatment cycle, twice in a kinase inhibitor treatment cycle, or three times in a kinase inhibitor treatment cycle. In other embodiments plinabulin is administered once a day, twice a day, three times a day, four times a day, every other day, once a week, twice a week, three times a week, once every other week, once every two weeks, and once every three weeks.

### CDK Inhibitors

The CDK inhibitors for use as described can include any agent, including a small molecule or biological agent, that inhibits the activity of cyclin-dependent kinases. Specific CDK inhibitors that can be used as described herein include palbociclib (e.g., IBRANCE), abemaciclib (e.g., VERZENIO), and ribociclib (e.g., KISQALI). Accordingly, some embodiments include co-administration of plinabulin and abemaciclib.

In some embodiments, the dose of the CDK inhibitor used is the dose for which the CDK inhibitor is therapeutically effective as a single agent. In other embodiments, the dose of the CDK inhibitor used in the co-administration is lower than the therapeutically effective dose as a single agent.

In various embodiments, the dose of the CDK inhibitor administered is 0.1 mg to 4000 mg, 0.25 mg-3500 mg, 0.25 mg-3000 mg, 0.25 mg-2500 mg, 0.25 mg-2000 mg, 0.25 mg-1500 mg, 0.25 mg-1000 mg, 0.25 mg-600 mg, 0.5 mg-600 mg, 0.75 mg-600 mg, 1 mg-600 mg, 150 mg-600 mg, 200 mg-600 mg, 300 mg-600 mg, 0.5 mg-500 mg, 0.5 mg-200 mg, 0.75 mg-200 mg, 1.0 mg-100 mg, 1.5 mg-100 mg, 2.0 mg-100 mg, 3.0 mg-100 mg, 4.0 mg-100 mg, 1.0 mg-80 mg, 1.5 mg-80 mg, 2.0 mg-80 mg, 3.0 mg-80 mg, 4.0 mg-80 mg, 1.0 mg-60 mg, 1.5 mg-60 mg, 2.0 mg-60 mg, 3.0 mg-60 mg, or about 4.0 mg-60 mg.

In various embodiments, the CDK inhibitor is administered once a day, twice a day, three times a day, four times a day, every other day, once a week, once every two weeks, and once every three weeks.

In various embodiments, the dose of the CDK inhibitor palbociclib administered is from 25 mg to 125 mg, from 50 mg to 100 mg, or from 70 mg to 80 mg. In some embodiments, the dose of palbociclib is 75 mg. In some embodiments, the dose of palbociclib is 100 mg. In some embodiments, the dose of palbociclib is 125 mg. In some embodiments, palbociclib is administered once daily. In some embodiments, palbociclib is administered twice daily.

In various embodiments, the dose of the CDK inhibitor abemaciclib administered is from 1 mg to 400 mg, from 25 mg to 250 mg, from 50 mg to 200 mg, from 100 mg to 150 mg, or about 125 mg. In some embodiments, the dose of abemaciclib is 50 mg. In some embodiments, the dose of abemaciclib is 100 mg. In some embodiments, the dose of abemaciclib is 150 mg. In some embodiments, the dose of abemaciclib is 200 mg. In some embodiments, abemaciclib is administered once daily. In some embodiments, abemaciclib is administered twice daily.

In various embodiments, the dose of the CDK inhibitor ribociclib administered is from 50 mg to 600 mg, from 100 mg to 400 mg, from 150 mg to 350 mg, or from 200 to 300 mg. In some embodiments, the dose of ribociclib is 200 mg. In some embodiments, the dose of ribociclib is 400 mg. In some embodiments, the dose of ribociclib is 600 mg. In some embodiments, ribociclib is administered once a day. In some embodiments, ribociclib is administered twice daily. In some embodiments, ribociclib is administered three times daily.

### Pharmaceutical Compositions

In some embodiments, plinabulin and the CDK inhibitor are administered in the same pharmaceutical composition. In other embodiments, plinabulin and the CDK inhibitor are administered in separate pharmaceutical compositions.

Administration of the pharmaceutical compositions described herein can be via any of the accepted modes of administration including, but not limited to, orally, sublingually, buccally, subcutaneously, intravenously, intranasally, topically, transdermally, intradermally, intraperitoneally, intramuscularly, intrapulmonarilly, vaginally, rectally, or intraocularly. In some embodiments, the pharmaceutical compositions are administered orally and/or parenterally. In some embodiments, plinabulin is administered intravenously and the CDK inhibitor is administered orally.

In various embodiments, pharmaceutical compositions include the active agent (e.g., plinabulin and/or the CDK inhibitor) and one or more pharmaceutically acceptable carrier or excipient. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The compositions described herein are preferably provided in unit dosage form. As used herein, a “unit dosage form” is a composition containing an amount of a compound or composition that is suitable for administration to an animal, preferably mammal subject, in a single dose, according to good medical practice. The preparation of a single or unit dosage form, however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, although a single administration is not specifically excluded. The skilled artisan will recognize that the formulation does not specifically contemplate the entire course of therapy and such decisions are left for those skilled in the art of treatment.

Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound or composition. The amount of carrier employed in conjunction with the compound or composition is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules (e.g., solid gel capsules and liquid gel capsules), granules and bulk powders. In some embodiments, the CDK inhibitor is provided in such an oral dosage form. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Oral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Oral liquid compositions may also contain one or more components such as sweeteners, flavoring agents and colorants disclosed above.

Such compositions may also be coated by conventional methods, typically with pH or time-dependent coatings, such that the subject composition is released in the gastrointestinal tract in the vicinity of the desired application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the composition disclosed herein are employed. Topical formulations may generally be comprised of a pharmaceutical carrier, co-solvent, emulsifier, penetration enhancer, preservative system, and emollient.

For intravenous administration, the compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration.

In some embodiments, the plinabulin for intravenous administration is provided in a pharmaceutical composition including one or more pharmaceutically acceptable diluents. In some embodiments, the pharmaceutically acceptable diluent can include Kolliphor® (Polyethylene glycol (15)-hydroxystearate). In some embodiments, the pharmaceutically acceptable diluent can include propylene glycol. In some embodiments, the pharmaceutically acceptable diluents can include Kolliphor (Kolliphor HS 15) and propylene glycol. In some embodiments, the pharmaceutically acceptable diluents can include Kolliphor and propylene glycol, wherein the Kolliphor is about 40% by weight and propylene glycol is about 60% by weight based on the total weight of the diluents. In some embodiments, the composition can further include one or more other pharmaceutically acceptable excipients. In some embodiments, a pharmaceutical composition can be diluted prior to administration, such as dilution with water, saline, or D5W (5% dextrose solution).

### Methods of Treatment

In some embodiments, plinabulin and a CDK inhibitor are co-administered to treat cancer. In various embodiments, the cancer comprises a homologous recombination repair deficient tumor. In some embodiments, the cancer comprises a mutation in a homologous recombination repair gene. In some embodiments, the cancer comprises a BRCA1 and/or BRCA2 mutation. In various embodiments, the cancer is an ovarian, breast, lung, pancreatic, fallopian, primary peritoneal, and prostate cancer. In some embodiments, the cancer is estrogen receptor positive (ER+) breast cancer. In some embodiments, the cancer is hormone receptor positive (HR+) breast cancer. In some embodiments, the cancer is progesterone receptor positive (PR+) breast cancer. In some embodiments, the cancer is HER2-breast cancer. In some embodiments, the cancer is HER2+ breast cancer. In various embodiments, the cancer is triple negative breast cancer. In some embodiments, the cancer is operable breast cancer. In some embodiments, the cancer is metastatic breast cancer. In some embodiments, the cancer is solid tumors. In other embodiments the cancer is metastatic pancreatic ductal adenocarcinoma (mPDAC). In some embodiments, the cancer is small cell lung cancer. In various embodiments, the cancer is non-small cell lung cancer (NSCLC). In some embodiments, the cancer is ovarian cancer. In some embodiments, the cancer is anaplastic lymphoma kinase (ALK+) cancer. In some embodiments, the cancer is head and neck squamous cell carcinoma (HNSCC). In other embodiments, the cancer is esophageal squamous cell carcinoma. In some embodiments, the cancer is gastrointestinal stromal tumor (GIST). In some embodiments, the cancer is colorectal cancer. In some embodiments, the cancer is endometrial cancer. In some embodiments, the cancer is glioblastoma. In various embodiments, the cancer is chronic lymphocytic leukemia (CLL). In some embodiments the cancer is a B-cell malignancies. In some embodiments, the cancer is acute myeloid leukemia.

In some embodiments, plinabulin is administered after administration of a CDK inhibitor. In other embodiments, plinabulin is administered before or simultaneously with a CDK inhibitor. In some embodiments, plinabulin is administered from 1 minute to 5 hours, 5 minutes to 4 hours, 10 minutes to 3.5 hours, 30 minutes to 3 hours, 30 minutes to 2 hours, 30 minutes to 1.5 hours, 1 hour to 2.5 hours, about 1 hour or about 2 hours after a CDK inhibitor. In some embodiments, plinabulin is administered from 1 minute to 5 hours, 5 minutes to 4 hours, 10 minutes to 3.5 hours, 30 minutes to 3 hours, 30 minutes to 2 hours, 30 minutes to 1.5 hours, 1 hour to 2.5 hours, about 1 hour or about 2 hours before a CDK inhibitor.

In some embodiments, plinabulin is administered on the same day that CDK inhibitor therapy is started. In other embodiments, plinabulin is administered on the day after, two days after, three days after, four days after, five days after, six days after, seven days after, eight days after, nine days after, and/or ten days after the start of CDK inhibitor therapy.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Examples

The anti-tumor activity of plinabulin as a single agent compared to co-administration with abemaciclib was evaluated in an MDA-MB-231 human breast cancer SubQ xenograft model in BALB/c nude mice.

Plinabulin was provided as a 4 mg/mL solution in 40% Solutol, 60% Propylene Glycol. Prior to use, this solution was diluted with D5W and gently mixed to achieve a clear solution of 0.75 mg/mL to deliver a dose of 7.5 mg/kg in a 10 mL/kg dosing volume.

Abemaciclib was provided as a crystalline solid. Prior to dosing, an appropriate amount of abemaciclib solid was weighed out and dissolved in 1% hydroxyethyl cellulose in 25 mM phosphate buffer, pH=2, to a concentration of 5 mg/mL to deliver a dose of 50 mg/kg in a 10 mL/kg dosing volume.

The vehicle control was 8% Kolliphor (Solutol) HS 15, 12 Propylene Glycol, and 80% D5W, formulated fresh prior to each dose. The vehicle control was dosed intraperitoneally at a 10 mL/kg dose volume.

The MDA-MB-231 human breast tumor cell line was maintained in L-15 medium supplemented with 10% FBS at 37° C. in an atmosphere of 100% air. Cells were sub-cultured upon reaching about 90% confluence by trypsin-EDTA treatment. Cells in an exponential growth phase were harvested using a trypsin-EDTA mixture.

Female BALB/c nude mice were received at 6-8 weeks of age. All mice were acclimated for 7 days prior to study initiation. The mice were housed in housed in polycarbonate cages and maintained under specific pathogen-free conditions. The mice were fed irradiated dry granule food and sterile drinking water was freely available.

Mice were inoculated subcutaneously on the right flank with 0.1 mL of L-15 medium/Martigel (1:1) mixture containing a suspension of 1×10⁷ cells/mouse of live MDA-MB-231 tumor cells.

Tumor bearing animals were monitored and tumors were measured periodically until they reached the designated start size of about 100-125 mm³. Twenty-eight days following inoculation, thirty-two mice with tumor sizes of 101-124 mm³ were randomized into four groups of eight mice, each with a mean tumor size of 114 mm³. Tumor volumes and body weights were recorded when the mice were randomized and were taken twice weekly thereafter. Clinical observations were made daily. Dosing was performed via intraperitoneal injection for vehicle control and plinabulin and administered via oral gavage for abemaciclib as described below in Table 1. Plinabulin was administered one hour post dose of abemaciclib on combined dosing days.

TABLE 1
Study Groupings

| | | Vehicle Control | Abemaciclib | Plinabulin |
|---|---|---|---|---|
| Group | N | (BIW* to end) | (QD* to end) | (BIW* to end) |
| 1. Vehicle Control (IP) | 8 | X | | |
| 2. Abemaciclib 50 mg/kg (PO) | 8 | | X | |
| 3. Plinabulin 7.5 mg/kg (IP) | 8 | | | X |
| 4. Abemaciclib 50 mg/kg | 8 | | X | X |
| (PO) + Plinabulin | | | | |
| 7.5 mg/kg (IP)** | | | | |

*BIW = biweekly; QD = daily
**Plinabulin was administered 1 hour post dose of abemaciclib on combined dosing days.

Mice will be euthanized as they reach an individual tumor volume endpoint of greater than or equal to 1,200 mm3. The study will be ended on Day 60 and all remaining mice that did not reach the tumor volume endpoint will be euthanized as long-term survivors.

At individual mouse endpoints or the study end, mice will be euthanized and tumors excised. Wet weights of the tumors will be recorded, and the tissues discarded.

Increase in survival fractions will be confirmed by the log rank test with a comparison of each treatment group to the Vehicle Control group. All animals that reach an individual tumor volume endpoint or are sacrificed as long-term survivors (LTS) are included in the statistical analyses. These data will be used to calculate mean and median survival and perform log rank comparisons for each group. For statistical analysis purposes, any mouse sacrificed as LTS will be assigned a death day of Day 60.

Mean TGI was calculated on Day 38 using the following formula. All mice are included in the TGI calculations.

$$TGI = [ 1 - \frac{( \overline{X} {Treated}_{( final )} - \overline{X} {Treated}_{( Day 1 )} )}{( \overline{X} Vehicle {Control}_{( final )} - \overline{X} Vehicle {Control}_{( Day 1 )} )} ] \times 100 \%$$

All statistical analyses in the xenograft study are performed with GraphPad Prism software. p≤0.05 was considered statistically significant.

Differences in Day 38 tumor volumes at selected points and percent change in body weight values were confirmed using a one-way ANOVA, unpaired parametric with the Tukey's Multiple Comparison test. A two-tailed Student's t-test with Welch's correction was also used to verify any differences between each group and the vehicle control, as well as single agents and their respective combination groups.

The Vehicle Control group [Group 1] resulted in a mean tumor volume of 1235±24 mm³ on Day 38. Treatment with abemaciclib (50 mg/kg) [Group 2] resulted in a mean tumor volume of 572±101 mm³. This group produced a TGI of 59% when compared to the Vehicle Control group on Day 38. Treatment with plinabulin alone 7.5 mg/kg [Group 3] resulted in a mean tumor volume of 942±114 mm³. This group produced a TGI of 26% when compared to the Vehicle Control group on Day 38.

Treatment with abemaciclib 50 mg/kg+plinabulin 7.5 mg/kg [Group 4] resulted in a mean tumor volume of 379±80 mm³ on Day 38. This group produced a TGI of 76% when compared to the Vehicle Control group on Day 38. A statistically significant decrease in mean tumor volume was observed when compared to abemaciclib alone on Days 38 and 40 (ANOVA; p<0.05).

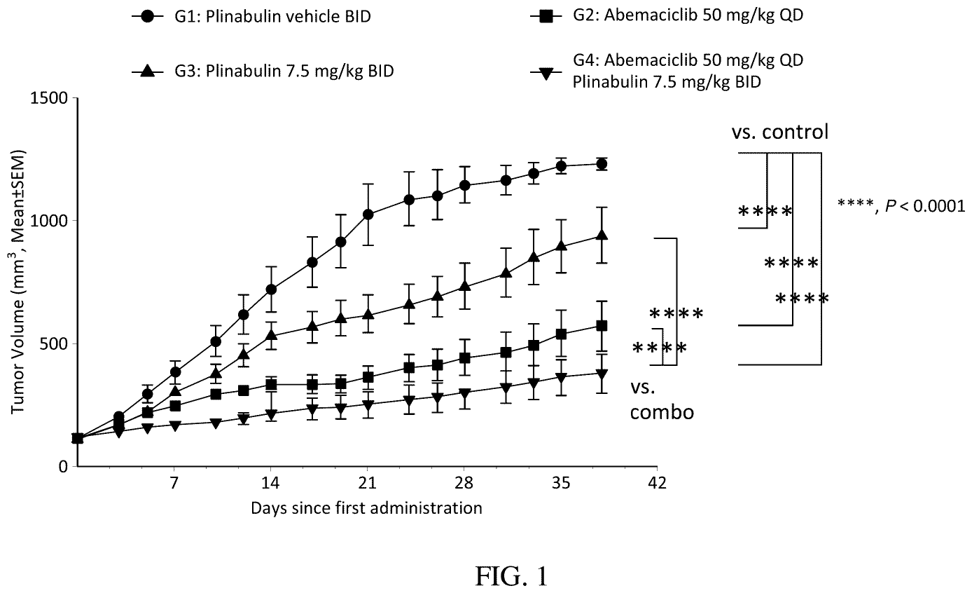

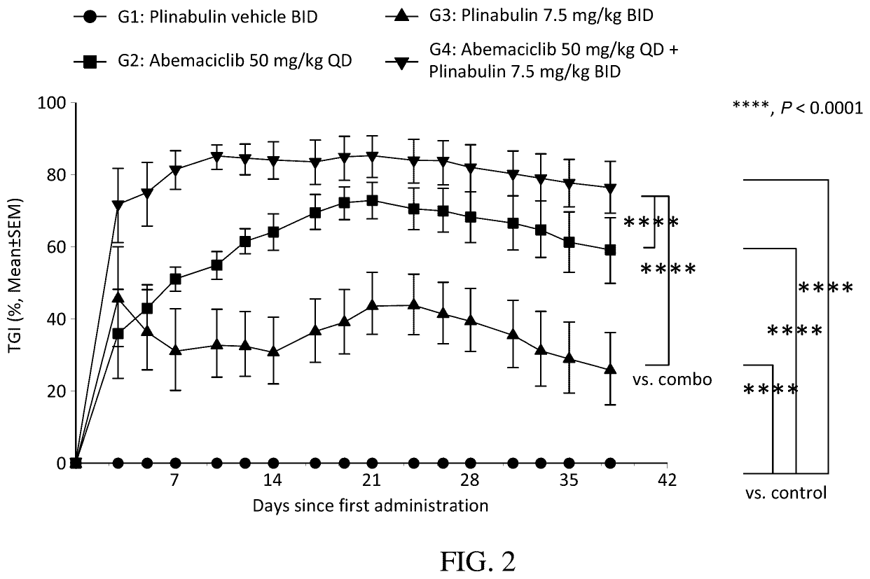

The effect of the various treatments on mean tumor volume (with last tumor volume carried forward) is shown in FIG. 1. Notably, as shown in FIG. 2, the combination therapy (Group 4) resulted in the greatest efficacy with a TGI of 76% on Day 38. Single treatment with abemaciclib (Group 2) or plinabulin (Group 3) produced TGIs of 59% and 26%, respectively on Day 38.

These results demonstrate the improved efficacy of the combination of abemaciclib and plinabulin.

What is claimed is:

1. A method of treating cancer, comprising co-administering to a subject in need thereof plinabulin and a cyclin-dependent kinase inhibitor.

2. The method of claim 1, wherein plinabulin is administered intravenously.

3. The method of claim 1 or 2, wherein the cyclin-dependent kinase inhibitor is administered orally.

4. The method of any one of claims 1-3, wherein the cyclin-dependent kinase inhibitor inhibits cyclin-dependent kinase 4 and/or cyclin-dependent kinase 6.

5. The method of any one of claims 1-4, wherein the cyclin-dependent kinase inhibitor is selected from the group consisting of palbociclib, ribociclib, and abemaciclib.

6. The method of any one of claims 1-5, wherein the cyclin-dependent kinase inhibitor is abemaciclib.

7. The method of claim 6, wherein abemaciclib is administered at a dose of 25 mg to 400 mg.

8. The method of claim 6 or 7, wherein abemaciclib is administered once per day.

9. The method of claim 6 or 7, wherein abemaciclib is administered twice per day.

10. The method of any one of claims 1-9, wherein the dose of plinabulin administered is from 10 mg/m² to 40 mg/m².

11. The method of any one of claims 1-9, wherein the dose of plinabulin administered is from 15 mg to 96 mg.

12. The method of claim 11, wherein the dose of plinabulin administered is about 40 mg.

13. The method of any one of claims 1-12, wherein plinabulin is administered once per week.

14. The method of any one of claims 1-12, wherein plinabulin is administered twice per week.

15. The method of any one of claims 1-12, wherein plinabulin is administered once every three weeks.

16. A pharmaceutical composition, comprising plinabulin or a pharmaceutically acceptable salt thereof and a cyclin-dependent kinase inhibitor or a pharmaceutically acceptable salt thereof.

17. The pharmaceutical composition of claim 16, wherein the cyclin-dependent kinase inhibitor inhibits cyclin-dependent kinase 4 and/or cyclin-dependent kinase 6.

18. The pharmaceutical composition of claim 16 or 17, wherein the cyclin-dependent kinase inhibitor is selected from the group consisting of palbociclib, ribociclib, and abemaciclib.

19. The pharmaceutical composition of any one of claims 16 to 18, wherein the cyclin-dependent kinase inhibitor is abemaciclib.

20. A kit, comprising:
a first pharmaceutical composition comprising plinabulin or a pharmaceutically acceptable salt thereof; and
a second pharmaceutical composition comprising a cyclin-dependent kinase inhibitor.

21. The kit of claim 20, wherein the first pharmaceutical composition is an intravenous formulation and the second pharmaceutical composition is an oral formulation.

22. The kit of claim 20 or claim 21, wherein the cyclin-dependent kinase inhibitor inhibits cyclin-dependent kinase 4 or cyclin-dependent kinase 6.

23. The kit of any one of claims 20 to 22, wherein the cyclin-dependent kinase inhibitor is selected from the group consisting of palbociclib, ribociclib, and abemaciclib.

24. The kit of any one of claims 20 to 23, wherein the cyclin-dependent kinase inhibitor is abemaciclib.
